(19) United States
(12) Patent Application Publication
(10) Pub. No.: US 2022/0093224 A1
(43) Pub. Date: Mar. 24, 2022

(54) Machine-Learned Quality Control for Epigenetic Data

(71) Applicant: Foxo Labs, Inc., Minneapolis, MN (US)

(72) Inventors: Nichole Rigby, Minneapolis, MN (US); Marc M. Maxmeister, Minneapolis, MN (US); Randal S. Olson, Minneapolis, MN (US); Brian H. Chen, Minneapolis, MN (US)

(21) Appl. No.: 17/482,405

(22) Filed: Sep. 22, 2021

Related U.S. Application Data
(60) Provisional application No. 63/082,427, filed on Sep. 23, 2020.

(51) Int. Cl. G16H 10/40; G16H 10/60; G16H 50/20; G16H 50/70; G16H 40/20; G16B 20/00

(52) U.S. Cl. CPC G16H 10/40; G16H 10/60; G16B 20/00; G16H 50/70; G16H 40/20; G16H 50/20

(57) ABSTRACT

High throughput quality control for epigenetic profiles associated with different subjects may include a pipeline that identifies as faulty epigenetic profile(s) from among a batch of epigenetic profiles and may additionally or alternatively includes a machine-learning (ML) model trained to identify the type of fault or condition that caused an epigenetic profile to be faulty. Such an ML model and techniques for training such an ML model are discussed herein.

This application claims the benefit of U.S. Provisional Application No. 63/082,427, filed Sep. 23, 2020, the entirety of which is incorporated by reference herein.

### BACKGROUND

Epigenetic data may be extracted from a biological sample collected from an individual. Any of a myriad of factors may taint epigenetic data, causing the data to be inaccurate and therefore unusable or unknowingly corrupted. Other factors may skew epigenetic data. Without identifying inaccurate or skewed epigenetic data, any processes which rely on such data may be frustrated, which may endanger or worry an individual that receives a diagnosis or therapy based on the epigenetic data or prevent the individual from obtaining an insurance or other longevity product that is are dependent on the epigenetic data, among other examples. Identifying inaccurate epigenetic data may be impossible for a human without the help of a machine and difficult and/or inaccurate with current quality control methods, particularly for laboratories that process hundreds or thousands of samples in a batch. Current quality control methods may only make rough estimates, resulting in discarding good samples and failing to discard bad samples.

### BRIEF DESCRIPTION OF THE DRAWINGS

The detailed description is described with reference to the accompanying figures. In the figures, the left-most digit(s) of a reference number identify the figure in which the reference number first appears. The same reference numbers in different figures indicate similar or identical items.

FIG. 1 illustrates a pictorial flow diagram of an example process for determining, by an ML model, an estimated health risk classification, biomarker status and/or level, and/or medical condition using epigenetic data associated with a subject.

FIG. 2 illustrates a block diagram of an example system that implements the techniques discussed herein.

FIG. 3 illustrates a first quality control technique for identifying epigenetic data as valid or invalid.

FIG. 4 illustrates a pictorial flow diagram of a second quality control technique comprising an example process for detecting faulty epigenetic data.

FIG. 5 illustrates a depiction of the embeddings (or a projection of the embeddings) determined as a part of the example process of FIG. 4.

FIGS. 6A-6F illustrate different clusters of distributions of epigenetic profiles.

FIG. 7 illustrates a graphical user interface for causing operations associated with different distributions of epigenetic profiles.

FIG. 8 illustrates a flow diagram of an example process for detecting that an epigenetic profile is faulty, detecting a fault associated with the epigenetic profile, and/or taking action regarding the fault.

FIG. 9 depicts a pictorial flow diagram of an example process 900 for training the embedding algorithm and/or clustering algorithm.

### DETAILED DESCRIPTION

The techniques (e.g., machine(s) and/or process(es)) discussed herein generally relate to quality control for epigenetic profiles associated with different subjects. An epigenetic profile may be an indication of the state of epigenetics of a subject and may be derived from a biological sample collected from the subject. For example, an epigenetic profile may comprise a measurement of methylation, acetylation, histone, and/or other similar modifications to DNA, RNA, or proteins of a subject. In some examples, the epigenetic profile may be acquired from a biological sample received from the subject. The biological sample may comprise saliva and/or blood, although it is contemplated that other samples may be collected from the subject. The epigenetic profile may comprise hundreds, tens of thousands, or even hundreds of thousands of indications of the epigenetic state (methylation, acetylation, histone/other modification), at different locations in the subject's DNA. For example, depending on the test equipment used, a subject's epigenetic profile will include a state of 27,578 locations in the subject's DNA. However, other test equipment results in determining the epigenetic state of a subject's DNA at 450,000 or beyond 850,000 locations in the DNA.

Moreover, a laboratory that processes epigenetic data for scientific research, medical diagnoses, medical treatment plan, or obtaining insurance or other longevity products may process hundreds or thousands of biological samples and/or epigenetic profiles a day. Identifying faulty samples/epigenetic profiles is therefore an exceedingly complex and tedious problem—so much so that prior quality control attempts have glossed over the issue by using inexact methods that roughly identify epigenetic profiles that are most likely not faulty. This lack of precision hampers the accuracy of scientific research, medical diagnoses, medical treatment plans, and/or correctly assigning longevity products to a subject.

The techniques discussed herein provide high-throughput quality control for epigenetic profiles. For example, the techniques may be used to accurately identify faulty epigenetic profiles from among a batch of hundreds, thousands, or even tens of thousands of epigenetic profiles at one time. The techniques may include a pipeline that identifies as faulty epigenetic profile(s) from among a batch of epigenetic profiles and may additionally or alternatively includes a machine-learning (ML) model trained to identify the type of fault or condition that caused an epigenetic profile to be faulty. For example, the ML model may receive as input an epigenetic profile that was identified by a first pipeline as being faulty and the ML model may be trained to output a classification associated with the faulty epigenetic data identifying one or more faults or conditions associated with the faulty epigenetic profile. Such a fault or condition may be associated with the collection, preservation, or processing of a biological sample from which the epigenetic data was derived. This ML model and/or pipeline identifies faulty epigenetic profiles with more accuracy and precision than former techniques and also automates the process, whereas former techniques required human manual delineation and subjective judgment.

For example, a fault or condition output by the ML model in association with a faulty epigenetic profile may indicate that the subject consumed or used mouthwash, toothpaste, alcohol, food, or the like before providing the biological sample from which the faulty epigenetic profile was derived; there was a failure to maintain an environmental temperature associated with storage of the epigenetic profile below a maximum environmental temperature; a maximum temperature associated with locations through which the first epigenetic profile was transported; a sample processing error; and/or a diagnosis of oral, laryngeal, esophageal, or lingual cancer associated with the subject, causing a skew of the epigenetic data.

In some examples, the ML model may additionally or alternatively regress a skew factor associated with the condition or fault. For example, cancer cells may skew an epigenetic profile and the ML model may output a factor that may be used to post-process the epigenetic profile to recover a non-cancer-skewed epigenetic profile that may be used for evaluating other states associated with the subject that may have otherwise been obscured by the skewing caused by the cancer (e.g., such as pharmaceutical or illicit drug use, a disease or biological health state, an actuarial category of risk associated with the subject).

The techniques may additionally or alternatively comprise causing a notification and/or instructions to be displayed via a user interface of a computing device associated with the subject or personnel that processed the biological sample. The notification and/or instructions may include a notification that a new sample collection device is being shipped to the subject, a tracking number associated with shipping the collection device, instructions for modifying provision of the new biological sample (e.g., “Please wait at least two hours after brushing your teeth or using mouthwash to provide the sample,” “The first sample provided was faulty due to [fault or condition]”), instructions for modifying a method for processing a biological sample, etc.

Some fault and/or conditions may be associated with epigenetic data restoration/or recovery processing operations. For example, if the ML model classifies a faulty epigenetic profile as being associated with a biological sample processing error and/or data processing error, the biological sample may be re-processed; or, in some instances, a subset of the epigenetic profile may be preserved while a second subset may be discarded. In some examples, a component of the pipeline and/or the ML model may identify a subset of DNA loci associated with the epigenetic profile that are associated with valid data. In such an example, the component and/or the ML model may output that subset, discard a faulty subset, and/or determine whether the valid subset is associated with a sufficient number or particular DNA loci to conduct meaningful downstream operations. For example, the downstream operations may include scientific research, medical diagnoses, medical treatment plan, and/or obtaining insurance or other longevity products and such operations may be based on particular DNA loci. In such an instance, the valid subset may still be used if the subset is sufficient for the post-processing operations.

The techniques discussed herein may improve epigenetic profile processing throughput (e.g., the number of epigenetic profiles that may be produced in a given amount of time), the number of good epigenetic profiles that are preserved from culling, and the quality of the epigenetic profiles that are output by the quality control techniques discussed herein (e.g., the number of faulty epigenetic profiles may be reduced). Moreover, the techniques may reduce lag between culling a biological sample by a quality control process and receiving a new biological sample, identify formerly unidentified reasons for failure to obtain a valid epigenetic profile associated with a specific subject, and/or expose latent reasons that an epigenetic profile is faulty. For example, the formerly unidentified reasons for invalid epigenetic profiles may include cross-contamination due to eating before obtaining the biological sample, mishandling the biological sample, drinking coffee or alcohol before obtaining the biological sample, the presence of cancer or another health condition in the subject, or the like.

The term, “array” or “microarray” as used herein means a tool designed to detect the presence of specific genetic sequences or alleles (some may represent methylated or unmethylated cytosines) in a plurality of genomic regions at the same time via the use of a plurality of probes that are fixed at set positions on a solid surface. The term, “probes” as used herein means a sequence of nucleic acids that have base pair complementarity to a sequence of interest. The term, “sequencing” as used herein means the process of determining a nucleic acid sequence through a variety of possible methods and technologies.

### Example Operations

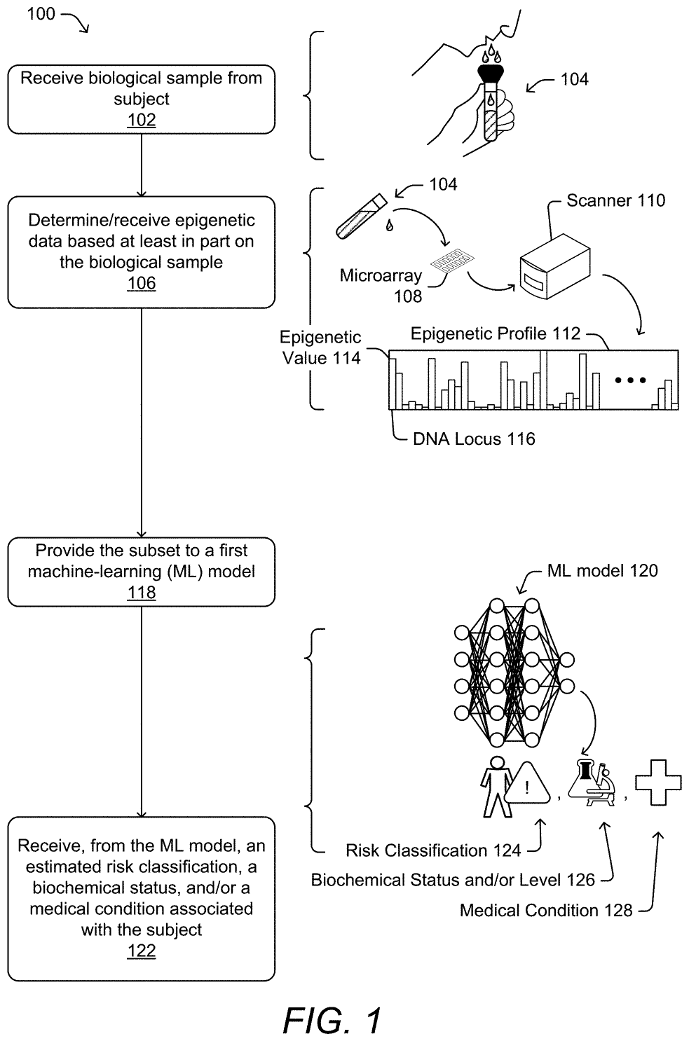

FIG. 1 depicts a pictorial flow diagram of an example process 100 for determining, by an ML model, an estimated risk classification, biomarker status and/or level, and/or medical condition using an epigenetic profile associated with a subject. In some examples, the quality control techniques discussed herein may include one or more operation(s) of example process 100. Note that FIGS. 1, 4, and 8 illustrate example processes in accordance with embodiments of the disclosure. These processes are illustrated as logical flow graphs, each operation of which represents a sequence of operations that can be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described operations can be combined in any order, in parallel to implement the processes, and/or may be performed as independent processes.

At operation 102, example process 100 may comprise receiving a biological sample 104 from the subject, according to any of the techniques discussed herein. For example, the biological sample may comprise one or more samples of tissue (e.g., skin, muscle, bone, adipose tissue, microbiome) and/or one or more samples of bodily fluid (e.g., saliva, whole blood, serum, plasma). The biological sample 104 may comprise DNA and/or be selected such that the biological sample 104 is likely to comprise DNA. In some examples, DNA containing biological samples can be obtained from an individual via saliva collection, as depicted in FIG. 1, although any other method of biological sample collection may be performed, such as a blood draw.

In some examples, receiving the biological sample may additionally or alternatively comprise isolating cells of the biological sample 104 by individual cell types. For example, the different cell types may include stem cells, erythrocytes, granulocytes (e.g., neutrophils, eosinophils, basophils), agranulocytes (e.g., monocytes, lymphocytes), platelets, neurons, neuroglial cells, skeletal muscle cells, cardiac muscle cells, smooth muscle cells, chondrocytes, keratinocytes, osteoclasts, osteoblasts, melanocytes, Merkel cells, Langerhans cells, endothelial cells, epithelial cells, adipocytes, spermatozoa, ova, and/or the like.

At operation 106, example process 100 may comprise determining and/or receiving an epigenetic profile associated with the subject based at least in part on the biological sample 104, according to any of the techniques discussed herein. For example, determining the epigenetic profile may comprise processing the biological sample 104 by 1) extracting genomic DNA from cells (e.g., epithelial cells, white blood cells) present in the biological sample 104 via one of several methods (e.g., salting out, phenol-chloroform, silica gel, benzyl-alcohol, magnetic beads), 2) denaturing the extracted DNA, 3) incubating the denatured DNA in a bisulfite containing compound and thermocycling at set temperatures and intervals, 4) purifying, desulphonating, and/or neutralizing of the bisulfite converted genomic DNA, and/or 5) measuring the methylation levels through DNA sequencing and/or microarray analysis. In general, this process of bisulfite conversion followed by array profiling or sequencing differentiates and detects unmethylated versus methylated cytosines by converting unmethylated cytosine base pairs to uracil, while methylated cytosines remain. In additional or alternative examples, the DNA may not undergo bisulfite conversion and may be analyzed without alteration (steps 3-4).

In at least one example, DNA prepared according to steps 1-4 may be applied to a microarray, such as array 108, which may comprise a fluorophore-, silver-, and/or chemiluminescent-labeled probe for indicating successful/failed binding of the probe to a particular DNA locus, which may indicate that the DNA locus is methylated/unmethylated and/or a level of methylation associated with the DNA locus. For example, array 108 may comprise a microarray chip (e.g., Illumina MethylationEPIC BeadChip (850 k array), IlluminaHumanMethyalation450 BeadChip (450 k array), IlluminaHumanMethyalation27 BeadChip (27 k array), chromatin immunoprecipitation (ChIP) microarray), a DNA chip, or a biochip. A scanner 110 may detect and quantify an intensity of the classification output (e.g., an intensity of the fluorescence associated with a probe) to determine a binary (unmethylated/methylated) or continuous (methylation level) indication of methylation at the DNA locus. In an additional or alternate example, any other method of processing the DNA may be performed (e.g., methylation specific polymerase chain reaction, methyl-sensitive cut counting, luminometric methylation assay, pyrosequencing).

In an additional or alternate example, processing the biological sample may comprise TET-assisted pyridine borane sequencing (TAPS) to determine the epigenetic profile.

Although the discussion herein discusses methylation data, it is understood that other epigenetic data such as, for example, acetylation, histone protein modifications, phosphorylation, sumoylation, and/or the like may be used to train the ML model(s) discussed herein (e.g., the ML model(s) may receive methylation data as input or any other type of epigenetic data). Epigenetic alterations to the genome comprise a host of different biochemical changes to DNA and proteins associated with it. For example, the cytosine residues of DNA may be methylated, which when methylated near regions that control gene expression (e.g., promoter regions), may alter gene expression. Another example is the modification of lysine tails on histone proteins. DNA wraps around histone proteins to form its superstructure. The modification of these tails may change the confirmation of DNA to make it more or less accessible for transcription and gene expression. These examples are not exhaustive and more are contemplated.

Regardless of what type of epigenetic data is obtained or used herein, FIG. 1 depicts an example representation of an epigenetic profile 112, determined by whatever means, which may comprise an epigenetic value 114 associated with a DNA locus 116. The depicted example shows additional epigenetic values associated with other DNA loci as well. Note that, as used herein, an epigenetic profile may include the epigenetic state (e.g., as reflected by the values discussed herein) at one or more DNA loci associated with a subject. For example, where an assay is used to determine the epigenetic profile 112 associated with a subject, the number of DNA loci (and the epigenetic states associated therewith) may depend on the assay—over 850,000 for an 850 k probe, over 450,000 for a 450 k probe, and so on.

In some examples, the epigenetic values of the epigenetic profile 112 may be normalized (e.g., quantile normalization, subset-quantile within array normalization (SWAN), beta-mixture quantile method, dasen, normal-exponential out-of-band (NOOB), single sample NOOB (ssNOOB)) and/or otherwise pre-processed (e.g., background correction, dye bias correction, pre-filtered to remove epigenetic values and DNA loci associated with n % of samples having detection p-values above a detection threshold, e.g., 0.05, 0.001, 0.0001, wherein n is a positive integer, e.g., 50, 30, 75). In some examples, the epigenetic value 114 may be a binary indication of methylation or unmethylation, or the epigenetic value 114 may comprise a Beta-value and/or M-value. Epigenetic “value” is also referred to herein as an epigenetic level and may reflect an epigenetic state at a particular DNA locus, such as the methylation status or level at a particular DNA locus. A Beta-value may be the ratio of the methylated probe intensity and the overall intensity (sum of methylated and unmethylated probe intensities). For example, the Beta-value for an i-th DNA locus may be defined as:

$${β}_{i} = \frac{max ( {y}_{i , {meth}_{y}} , 0 )}{max ( {y}_{i} , {unmeth}_{y} , 0 ) + max ( {y}_{i , {meth}_{y}} , 0 ) + α} \qquad ( 1 )$$

where y_{i,methy} and y_{i,unmethy} are the intensities measured by the i-th methylated and unmethylated probes, respectively, and a is an offset (e.g., default value of 0 or 100) to regularize the Beta-value when the methylated and unmethylated probe intensities are low. A Beta-value of 0 indicates that all copies of the DNA locus in the sample were completely unmethylated and vice versa (full methylation of all copies of DNA locus in sample) for a Beta-value of 1.

The M-value may be the log 2 ratio of the intensities of methylated probe versus unmethylated probe. For example, the M-value for an i-th DNA locus may be defines as:

$${M}_{i} = {log}_{2} ( \frac{max ( {y}_{i , {meth}_{y}} , 0 ) + α}{max ( {y}_{i , {unmeth}_{y}} , 0 ) + α} ) \qquad ( 2 )$$

where α is an offset (e.g., default value of 1) to decrease the sensitivity of the equation to intensity estimation errors. An M-value close to 0 indicates a similar intensity between the methylated and unmethylated probes associated with a DNA locus, which means that the DNA locus may be approximately half methylated, assuming the intensity data is normalized. A positive M-value indicates more molecules are methylated than unmethylated, whereas the opposite is true of negative M-values.

The DNA locus 116 may be related to one or more DNA base pairs. For example, a DNA locus 116 may comprise an individual DNA base pair at a particular genomic location or may be a genomic region such as a CpG island, a promoter, an enhancer, an activator, a repressor, a transcription start site, and/or the like. While these regions are indicated, others are contemplated. For example, the DNA locus may comprise non-coding RNA molecules (nc-RNAs) such as micro RNA (miRNA), small interfering RNA (siRNA), piwi-interacting RNA (piRNA), and/or long non-coding RNA (lncRNA). These nc-RNAs may also be measured through sequencing, microarray, or polymerase chain reaction assays.

At operation 106, example process 100 may additionally or alternatively comprise the quality control techniques discussed herein and/or example process 100 may comprise adding the epigenetic profile to a batch of epigenetic profiles associated with multiple subjects. If the epigenetic profile 112 is culled from the batch based at least in part on the techniques discussed herein, example process may end, as regarding epigenetic profile 112 anyway, unless the epigenetic profile 112 is to undergo post-processing, re-processing, and/or a subset of the epigenetic profile 112 is identified as being valid. In the latter instance or if the epigenetic profile 112 is deemed valid according to at least some of the techniques discussed herein, example process 100 may continue to operation 118.

At operation 118, example process 100 may comprise providing at least a subset of epigenetic profile 112 as input to an ML model 120, according to any of the techniques discussed herein. For example, the ML model 120 may comprise, for example, a support vector machine (SVM) (e.g., Nystroem Kernel SVM, radial basis function (RBF) kernel SVM), a regression algorithm (e.g., ordinary least squares regression (OLSR), linear regression, logistic regression, Ridge regression, Lasso regression, ElasticNet regression), decision tree algorithms (e.g., classification and regression tree (CART), iterative dichotomiser 3 (ID3), Chi-squared automatic interaction detection (CHAID), decision stump, conditional decision trees, LightGBM, gradient-boosting machines (GBM), gradient boosted regression trees (GBRT), random forest), Bayesian algorithms (e.g., naïve Bayes, Gaussian naïve Bayes, multinomial naïve Bayes, average one-dependence estimators (AODE), Bayesian belief network (BNN), Bayesian networks), clustering algorithms (e.g., k-means, k-medians, expectation maximization (EM), hierarchical clustering), a neural network (e.g., a multilayer perceptron (MLP), ResNet50, ResNet101, ResNet 152, VGG, DenseNet, PointNet, a multi-task model that predicts multiple outcomes of one or more of risk classification, biomarker status and/or level(es), and/or medical condition(s), a multi-input model that receives epigenetic data associated with multiple biological sample types and/or assay types). In some examples, the ML model 120 may be learned according to supervised, unsupervised, or semi-supervised learning techniques. For example, the ML model 120 may be at least one of the ML models discussed in U.S. patent application Ser. No. 16/579,777, filed Sep. 23, 2019 and/or U.S. patent application Ser. No. 16/591,296, filed Oct. 2, 2019, each of which are incorporated in their entirety herein.

The techniques discussed herein may comprise different types of ML models and/or multiple ML Models of a same type that may have the same or similar hyperparameters. A parameter, in contrast to a hyperparameter, may comprise any parameter that is modified during training such as, for example, a weight associated with a layer or components thereof (e.g., a filter, node). Although various examples of hyperparameters are given herein, it is contemplated that one or more of the hyperparameters may be parameters, depending on the training method.

At operation 122, example process 100 may comprise receiving an estimated risk classification 124, a biomarker status and/or level 126, and/or a medical condition 128 associated with the subject, according to any of the techniques discussed herein. The ML model 120 may determine the estimated risk classification 124, biomarker status and/or level 126, and/or medical condition 128, based at least in part on the subset and a set of parameters (e.g., a weight, bias, y term, split points, and/or ϕ term associated with a node and/or layer of the ML model 120) associated with the ML model 120. As discussed above, the output may be indicated by a classification and/or a value. The output may additionally or alternatively comprise a confidence and/or confidence interval associated with the classification or value

In some examples, a risk classification 124 may comprise an actuarial classification, such as a life insurance underwriting risk category (e.g., Preferred Plus Non-Nicotine, Preferred Non-Nicotine, Standard Non-Nicotine, Standard Nicotine, Uninsurable) and/or a score associated with all-cause mortality (e.g., a prognostic score, a measure of estimated time until death, a hazard ratio, mortality factor). In some examples, the risk classification 124 may be used to determine eligibility for a level of coverage associated with a financial product (e.g., a level of health/life insurance coverage), an annuity amount (e.g., a pension payment, a loan where the loan is contingent on physical performance of the subject), and/or the like.

The techniques may comprise additional or alternate ML models trained to receive epigenetic data associated with the subject as input and to output a value associated with a biomarker status and/or level 126 and/or a value and/or classification associated with a medical condition 128. For example, a value associated with a biomarker status and/or level 126 may indicate a value associated with a level of alanine aminotransferase, albumin, alkaline phosphatase, anti-hepatitis B surface antigen, antibody to hepatitis C virus, apolipoprotein A1, apolipoprotein B, aspartate aminotransferase, bilirubin, total C-reactive protein, total cholesterol, CMV IgG, creatinine, cystatin C, gamma glutamyl transferase, globulin, glucose, HDL-cholesterol, hepatitis B surface antigen, HIV antigen/antibody combination, LDL-cholesterol, pro-brain natriuretic peptide, N-Terminal, total prostate-specific antigen, total protein, triglycerides, urea nitrogen, calcium, uric acid, very low-density lipoprotein, albumin, albumin:creatinine ratio, drug level (e.g., pharmaceutical or otherwise, amphetamine, barbiturate, benzodiazepine, cannabinoids, cocaine, opiate), ketones, leukocyte esterase, nitrite, blood/urine pH, phencyclidine, specific gravity, total protein per g creatinine ratio, urobilinogen, % or count of basophils in blood, % or count of eosinophils in blood, % or count of lymphocytes in blood, % or count of monocytes, % or count of neutrophils, platelet count (e.g., may include platelet distribution width), hematocrit, hemoglobin, hemoglobin A1c, ion-exchange HPLC, lymphocyte count, mean corpuscular volume, mean platelet volume, red blood cell count (e.g., may include red blood cell distribution width), white blood cell count, cotinine, 4-(methylnitrosamino)-1-(3-pyridyl)-1-butanol (NNAL), carbohydrate deficient transferrin (CDT), and/or Phosphatidyl Ethanol (PEth).

To give a non-limited example of a classification associated with a medical condition 128, the classification may comprise an indication of the presence or absence of alcohol use, arrhythmia (e.g., atrial fibrillation), asthma, cardiovascular condition (e.g., angina, angioplasty or percutaneous transluminal coronary angioplasty (PTCA), percutaneous coronary intervention (PCI), coronary artery bypass graft (CABG), coronary bypass surgery, state indicative of myocardial infarction (imminent or past), congestive heart failure), cancer (e.g., bladder, brain, breast, cervical, colon and/or rectum, endometrial, esophageal, kidney and/or renal pelvis, leukemia, liver, lung and bronchus, non-Hodgkin lymphoma, oral, pancreas, prostate, skin, stomach, thyroid, uterine), celiac status, chronic obstructive pulmonary disease (COPD) (e.g., bronchitis, emphysema), cognitive impairment (e.g., mild cognitive impairment (MCI)), cerebrovascular accident (CVA) (e.g., hemorrhagic stroke, ischemic stroke, transient ischemic attack (TIA)), dementia (Alzheimer's disease, frontotemporal disorders, Lewy body dementia, vascular dementia), diabetes (Type 1 and/or Type 2), drug abuse (e.g., illicit drug use, pharmaceutical abuse), epilepsy, hypertensive heart disease (e.g., hypertension, left ventricular hypertrophy), inflammatory bowel disease (e.g., Crohn's disease, ulcerative colitis), kidney disease (e.g., chronic kidney disease, end-stage renal disease), liver disease (e.g., cirrhosis), mental illness (e.g., anxiety, bipolar, depression, post-traumatic stress disorder (PTSD)), multiple sclerosis, osteoporosis, Parkinson's disease, arthritis (e.g. rheumatoid arthritis), symptomatic sensitivity and/or an identifier of the allergen, tobacco use, and/or cannabinoid use.

Without limitation, examples of values associated with a medical condition 128 may comprise an (average) number of alcoholic drinks per time period (e.g., week, year, drinking session), an (average) number of tobacco products per time period (e.g., week, year), a score indicating a stage of cancer, disease severity, a severity of symptomatic sensitivity, and/or the like.

In some examples, the risk classification 124, biomarker status and/or level 126, and/or medical condition 128 may comprise a discriminative indication (e.g., identifying a current condition) or a forecasting indication (e.g., identifying a likelihood of a future condition).

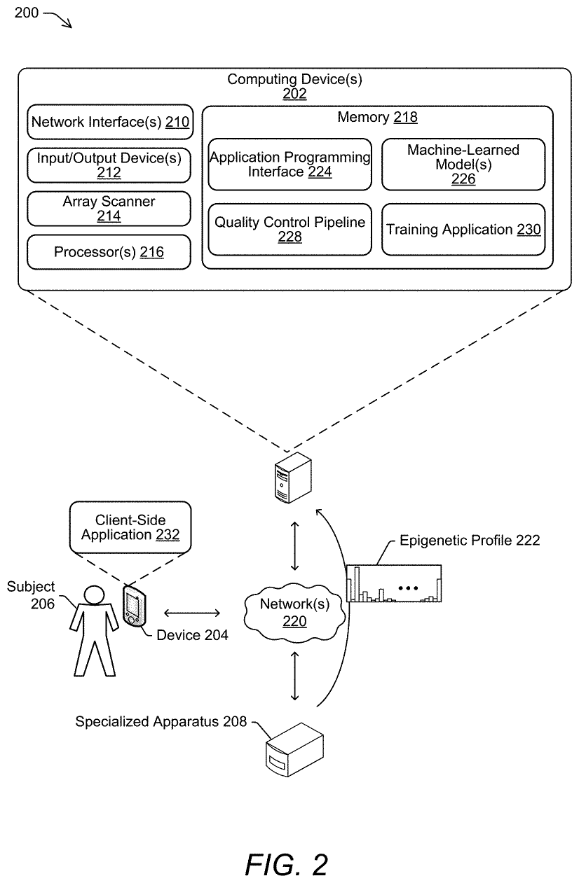

FIG. 2 depicts a block diagram of an example system 200 that implements the techniques discussed herein. In some instances, the system 200 may include service computing device(s) 202, a computing device 204 associated with a subject 206, and a specialized apparatus 208. In some examples, service computing device(s) 202 may comprise one or more nodes of a distributed computing system (e.g., a cloud computing architecture) although in additional or alternate examples, the service computing device(s) 202 may comprise a single server, multiple servers, and/or one or more computing device(s) suitable to accomplish the techniques discussed herein.

In some examples, the service computing device(s) 202 may comprise network interface(s) 210, input/output device(s) 212, an array scanner 214 (or other type of apparatus for determining epigenetic data from a DNA sample), processor(s) 216, and/or a memory 218.

Network interface(s) 210 may enable communication between the service computing device(s) 202 and one or more other local or remote computing device(s). For instance, the network interface(s) 210 may facilitate communication with other local computing device(s) of the service computing device(s). The network interface(s) 210 may additionally or alternatively enable the service computing device(s) 202 to communicate with computing device 204 and/or specialized apparatus 208 via network(s) 220.

The network interface(s) 210 may include physical and/or logical interfaces for connecting the service computing device(s) 202 to another computing device or a network, such as network(s) 220. For example, the network interface(s) 210 may enable Wi-Fi-based communication such as via frequencies defined by the IEEE 802.11 standards, short range wireless frequencies such as Bluetooth®, cellular communication (e.g., 2G, 3G, 4G, 4G LTE, 5G, etc.) or any suitable wired or wireless communications protocol that enables the respective computing device to interface with the other computing device(s). In some instances, the service computing device(s) 202 may transmit instructions via the network interface(s) 210 to an application running on the computing device 204 to cause operations at the device 204 as discussed herein and/or transmit instructions to the specialized apparatus 208 to cause the specialized apparatus to determine epigenetic profile 222 and/or transmit epigenetic profile 222 to the service computing device(s) 202.

In some instances, the service computing device(s) 202 may include input/output device(s) 212 for human and/or artificial intelligence (AI) interaction with the service computing device(s) 202 (e.g., an application programming interface between an AI component and the service computing device(s) 202, keyboard, mouse, display, biometric scanner, microphone, speaker, tactile response generator).

Additionally or alternatively, the service computing device(s) 202 may comprise an array scanner 214. The array scanner 214 may represent a device similar or identical to the specialized apparatus 208 and/or may be any other component for determining epigenetic data from a DNA sample. For example, the array scanner 214 may detect fluorescent, radioactive, or other labels indicative of methylation of a DNA locus probed by a microarray.

The service computing device(s) 202 may include one or more processors, processor(s) 216, and memory 218 communicatively coupled with the processor(s) 216. The processor(s) 216 may be any suitable processor capable of executing instructions to process data and perform operations as described herein. By way of example and not limitation, the processor(s) 216 may comprise one or more central processing units (CPUs), graphics processing units (GPUs), integrated circuits (e.g., application-specific integrated circuits (ASICs), etc.), gate arrays (e.g., field-programmable gate arrays (FPGAs), etc.), and/or any other device or portion of a device that processes electronic data to transform that electronic data into other electronic data that may be stored in registers and/or memory.

Memory 218 may be an example of non-transitory computer-readable media. The memory 218 may store an operating system and one or more software applications, instructions, programs, and/or data to implement the methods described herein and the functions attributed to the various systems. In various implementations, the memory may be implemented using any suitable memory technology, such as static random access memory (SRAM), synchronous dynamic RAM (SDRAM), nonvolatile/Flash-type memory, or any other type of memory capable of storing information. The architectures, systems, and individual elements described herein may include many other logical, programmatic, and physical components, of which those shown in the accompanying figures are merely examples that are related to the discussion herein.

In some instances, the memory 218 may store an application programming interface (API) 224, ML model(s) 226, quality control pipeline 228, and/or training application 230. In some examples, the API 224 may allow the device 204 or any other device (e.g., a device associated with a physician, laboratory, insurance company, and/or the like) to transmit calls to the service computing device(s) 202, causing the service computing device(s) 202 to conduct any of the operations discussed herein, and/or to receive responses from the service computing device(s) 202, which may comprise output(s) of the machine-learned model(s) 226 and/or determinations made according to the techniques discussed herein. In some examples, the API 224 may be based at least in part on the representational state transfer (REST) protocol, simple object access protocol (SOAP), and/or JavaScript. In some examples, the API 224 may allow computationally heavy or proprietary functions (e.g., such as the ML model(s), criteria, and/or hierarchies, and/or use thereof) to be stored remotely from users of the techniques described herein, while still providing data determined according to the techniques discussed herein to computing devices associated with the users. For example, the API 224 may transmit results of the operations discussed herein to device 204, specialized apparatus 208, and/or any other device.

For example, computing device 204 may execute a client-side application 232 that transmits requests to the API 224 and receives responses therefrom. In some examples, the responses may comprise the instructions discussed herein. In some examples, the client-side application may cause display of the UI discussed herein and may enable/disable selection of a coverage level and/or payment level associated with a financial product. In some examples, client-side application 232 may provide a UI that may be populated according to instructions transmitted by the service computing device(s) 202.

ML model(s) 226 may include and/or represent any of the ML models discussed herein and may comprise software, hardware (e.g., a circuit configured according to parameters determined during training), or a combination thereof (e.g., an FPGA configured as an ML model). The ML model(s) 226 discussed herein may comprise an embedding algorithm such as uniform manifold approximation and projection (UMAP), t-distributed stochastic neighbor embedding (t-SNE), ISO map, local linear embedding (LLE), or other such algorithm; and/or a classification algorithm such as, for example, a neural network, decision tree, Bayesian inference model, or the like. In some examples, the training application 230 may comprise software instructions for training the ML model(s) 226. In some examples, the ML model(s) 226 may comprise a first ML model for determining embeddings, a second ML model for determining a classification (e.g., valid epigenetic profile, invalid epigenetic profile, invalid epigenetic profile recoverable by a pre and/or post-processing operation), and/or a third ML model for determining a fault or condition associated with an invalid epigenetic profile. In some instances, the first, second, and/or third ML model may be combined into a single or more ML model(s) or divided into further sub-ML models.

The quality control pipeline 228 may comprise software, hardware, or a combination thereof for accomplishing the operations discussed herein. In some examples, the quality control pipeline 228 may comprise the ML model(s) 226 and/or one or more pre and/or post-processing components, such as a component that normalizes, skews, and/or isolates (e.g., selects particular probe locations from among multiple probe locations) epigenetic data, and/or the like.

It should be noted that while FIG. 2 illustrates components of service computing device(s) 202 as being within a single system the components may alternatively be stored, executed, or exist at different systems that may be communicatively coupled. Moreover, although a component may be depicted as being part of service computing device(s) 202, alternatively, the component may be stored, executed, or exist at device 204, specialized apparatus 208, and/or any other device.

### First Example Quality Control Technique

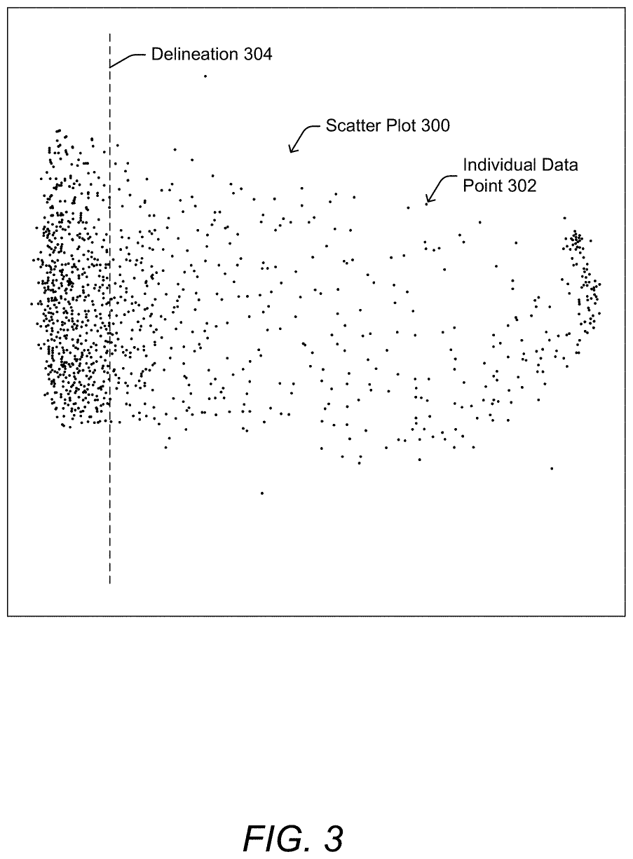

FIG. 3 illustrates a depiction of a first quality control technique for identifying epigenetic data as valid or invalid. The depicted example includes a scatter plot 300 comprising a plurality of data points where each data point represents a different epigenetic profile. An individual data point 302 may be derived from an epigenetic profile and may comprise a set of coordinates determined based at least in part on a decomposition of the epigenetic profile. The decomposition may be determined based at least in part on a principal component analysis or a similar such technique that reduces the epigenetic profile from a high-dimensional data set to a low-dimensional (e.g., 2, 3, 4) set of data. For example, decomposing the epigenetic profile may comprise determining a two-dimensional projection of the epigenetic profile, as represented by individual data point 302, using principal component analysis (PCA), multidimensional scaling (MDS), or the like.

The decomposed data points representing the epigenetic profiles may be plotted, as represented by the scatter plot in FIG. 3. According to the first quality control technique, a human may identify a set of valid epigenetic profile(s) using a line or other delineation 304. Generally, a human may look for a clump of data points on the left-hand side, which may represent epigenetic profiles that are valid. The first quality control technique thus identifies epigenetic profiles associated with the data points on the left side of the delineation 304 as being valid and the data points on the right side of the delineation 304 as being invalid. This process is entirely manual and subjective, based on human's best guess, and may therefore introduce a high degree of inaccuracy.

However, as this example illustrates, the first quality control technique may result in inadvertently identifying some epigenetic profiles as being invalid that are valid (false invalidation) and some other epigenetic profiles as being valid that are invalid (false validation). False validation may be a major detriment to any processes or machines that use the epigenetic profiles (e.g., medical diagnoses and/or treatment, scientific research, obtaining a correct longevity product). False invalidation may unnecessarily duplicate collection of a biological sample, result in the loss of financially and temporally expensive biological data that was otherwise valid and could have been used, etc.

### Example Quality Control Process

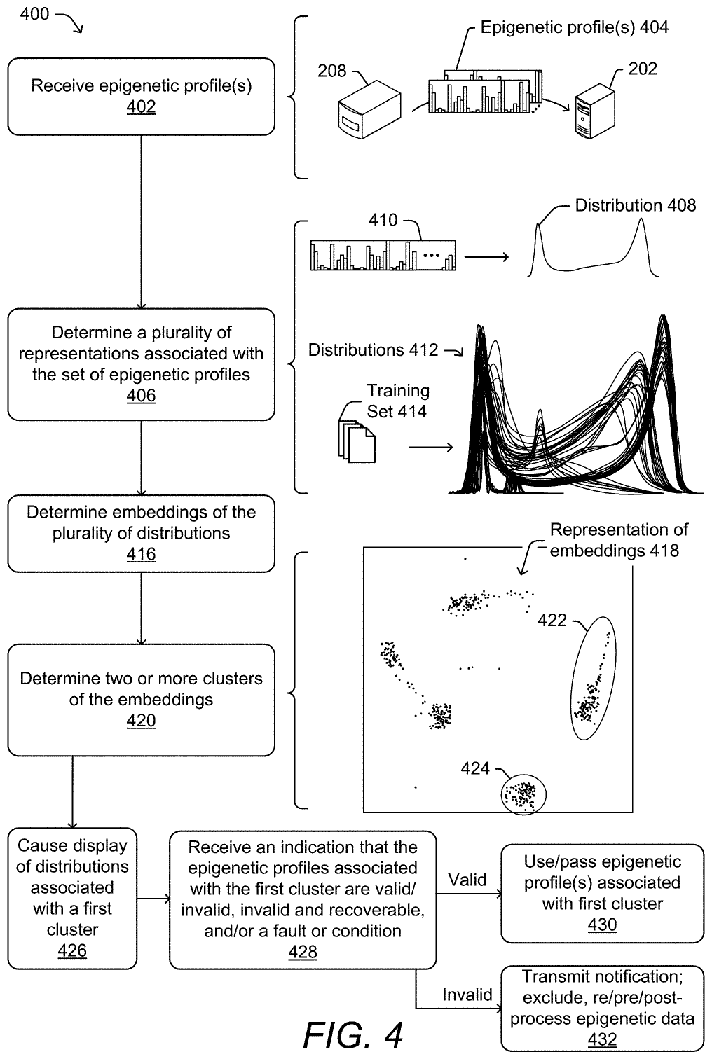

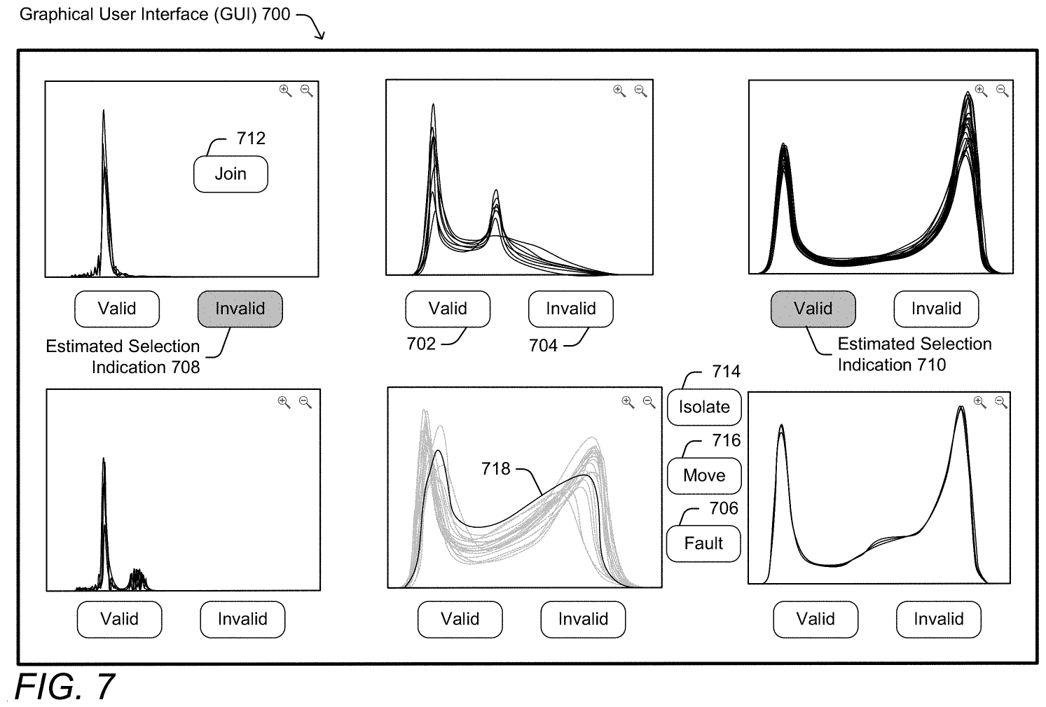

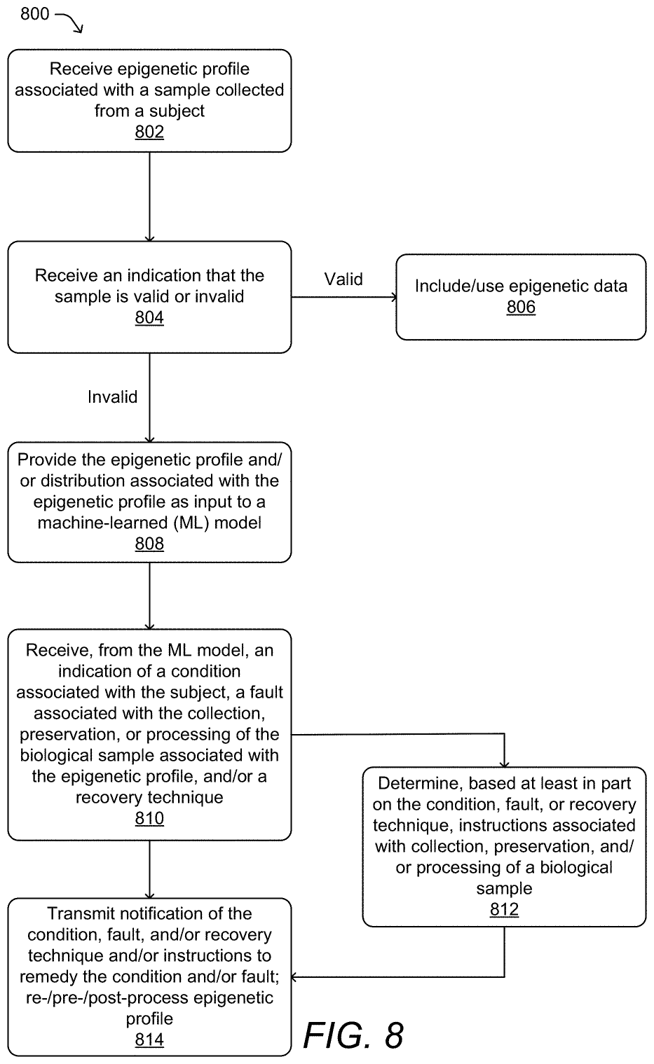

FIG. 4 depicts a pictorial flow diagram of an example process 400 for a second quality control technique for detecting faulty epigenetic profile(s). In some examples, example process 400 may be used as part of a training method for generating an ML model that detects faulty epigenetic profile(s) and/or as a quality control process executed by a computer. In an additional or alternate method, example process 400 may be used as part of a quality control pipeline for processing biological samples to generate epigenetic profiles for a variety of uses. Note that previous knowledge regarding whether an epigenetic profile is valid or invalid is not necessary to train the ML models discussed herein because of the process described in FIG. 4 and the training described in association with the GUI of FIG. 7. The techniques eschew the need for initial training data or supervision to detect invalid and/or valid epigenetic profiles.

At operation 402, example process 400 may comprise receiving one or more epigenetic profiles 404, according to any of the techniques discussed herein. Operation 402 may comprise receiving an epigenetic profile from a specialized apparatus 208, a computing device associated with a subject 206, and/or a data repository. In some examples, the service computing device(s) 202 may receive the epigenetic profile(s). In an additional or alternate example, an indication of how the epigenetic data was determined (e.g., a biological sample type, an array type) may be received in association with an epigenetic profile. In some examples, a specialized computing device, such as a scanner, may provide the indication in a metadata file associated with the epigenetic data. For example, the metadata file may indicate an algorithm by which values of the epigenetic data were generated, a biological sample type (e.g., saliva, blood, tissue), an array type (e.g., 450k, 850k), any errors generated by the scanner, or the like. In some examples, operation 402 may additionally or alternatively comprise a generic quality control operation, such as removing sub-standard probes or sample data and/or pre-processing operation(s), such as background subtraction, dye-bias correction, and/or normalization.

At operation 406, example process 400 may comprise determining a plurality of representations associated with the set of epigenetic samples, according to any of the techniques discussed herein. For example, operation 406 may comprise determining a distribution 408 associated with an individual epigenetic profile 410 and/or determining a plurality of distributions 412 associated with a training (set) 414 of epigenetic profiles. Determining the distribution 408 associated with an epigenetic profile may comprise determining an occurrence rate of bands of epigenetic values. For example, determining the distribution 408 may include determining a histogram of the epigenetic values associated with the epigenetic profile 410 and/or determining a kernel density estimate of the histogram or of the epigenetic values (e.g., M-values, beta-values) directly. In some examples, any method of estimating probability density may be used to smooth the epigenetic data associated with the epigenetic profile 410. In some examples, determining the distribution 408 may comprise smoothing the distribution.

In at least one example, determining the distribution 408 associated with a particular epigenetic profile may comprise determining binning epigenetic data associated with the epigenetic profile. For example, determining the distribution 402 may comprise generating 100 bins associated with different ranges of beta values and determining a proportion of probes of an array that have a value associated with one of the bins. For example, a first bin may be associated with beta-values between 0 to 0.0138756 and may be associated with a proportion (e.g., percentage, fraction) or count of probes having beta values that fall into a range between 0 to 0.0138756. More or less bins may be used (e.g., 10, 50, 150, 200, 1,000, 10,000).

In yet another example, an epigenetic profile may be transformed into an image or other representation comprising a shape representing epigenetic values of an epigenetic profile. The shape may be a line, such as the line depicted as representing distribution 408, a circular gain map similar to a radar cross section that associates different ranges of beta values with the count or proportion of probes having a beta value associated therewith, or any other suitable shape for representing the distribution 408. The epigenetic profile may additionally or alternatively comprise additional data such as, for example, DNA quality and/or quantity metrics (e.g., qPCR quantification of human and/or non-human DNA in the sample), test conditions, sample identification, and/or the like.

In an additional or alternate example, operation 406 may additionally or alternatively include determining some other representation of an epigenetic profile, such as a transformation or reduction of the high-dimensional epigenetic profile to a lower dimensionality. Dimensionality reduction techniques may be used, such as feature extraction, principal component analysis, linear discriminant analysis, canonical correlation analysis, or the like, and/or the epigenetic profile may be transformed into a line or set of lines and/or an image representing a distribution or reduced-dimensionality data set, in at least one example.

In some examples, the techniques discussed herein may be extensible to a multitude of biological sample types and assay types. Normally this may not be possible since different biological samples and assay types exhibit different epigenetic patterns and/or values, but operations 402, 406, 416, and/or 420 may extend the quality control techniques beyond assays and tissue samples. This is achieved, at least in part, by transforming the epigenetic values into a representation thereof, such as an image, line, or other representation of a distribution and/or using such representation to determine an embedding associated therewith.

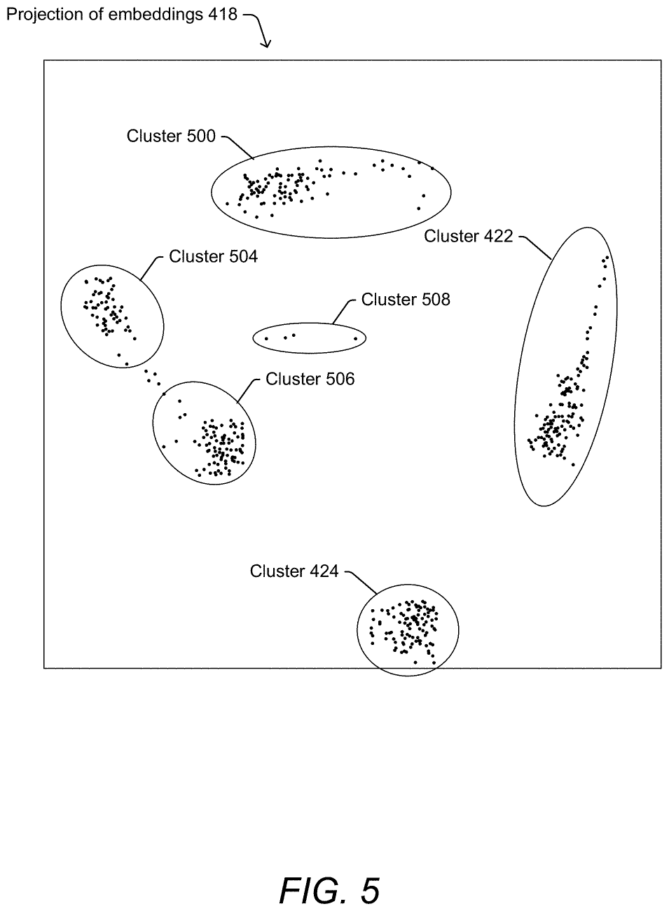

At operation 416, example process 400 may comprise determining embeddings of the plurality of distributions, according to any of the techniques discussed herein. In at least one example, operation 416 may comprise determining an embedding for each epigenetic profile and the embeddings may be determining by a machine-learned (ML) model. The ML model may comprise an embedding algorithm trained to map a distribution representing an epigenetic profile to a location (i.e., “embedding”) in an embedding space, which may be a high dimension space, such as a 32-dimensional, 256-dimensional, 512-dimensional or more-dimensional space. The location to which the ML model maps a distribution may be based at least in part on a similarity and/or dissimilarity of the distribution to other distributions. An embedding may be a high-dimensional representation of a location in the embedding space. FIG. 4 depicts a representation of multiple embeddings 418 as dots in a two-dimensional space that represent the embeddings generated based at least in part on the distributions 412, including distribution 408. FIG. 5 illustrates the representation 418 in greater size and detail.

Note that in order to achieve a representation, such as representation 418, the embeddings may need to be reduced from a high-dimensional tensor or vector to a two- or three-dimensional tensor or vector. This may comprise projecting an embedding from an embedding space into a two- or three-dimensional space. For example, the representation of embeddings 418 may have been generated by projecting the distributions 412 into a two-dimensional space.

In some examples, the ML model discussed herein may be trained to receive an epigenetic profile as input and may output an embedding associated with the epigenetic profile. In some examples, the ML model may additionally or alternatively output an indication of a cluster to which the embedding belongs although, in an additional or alternate example, clustering may be part of a subsequent operation of a separate algorithm. In some examples, the ML model may be based at least in part on an embedding algorithm. In at least one example, the embedding algorithm may be a manifold learning projection algorithm, such as uniform manifold approximation and projection (UMAP), t-distributed stochastic neighbor embedding (t-SNE), or other such algorithms.

At operation 420, example process 400 may comprise determining two or more clusters of the embeddings, according to any of the techniques discussed herein. Determining the two or more clusters of embeddings may comprise applying a clustering algorithm, such as density-based spatial clustering of applications with noise (DBScan), hierarchal density-based spatial clustering of applications with noise (HDB Scan), k-means, or the like to the embeddings determined at operation 416. FIG. 4 depicts a first cluster 422 of embeddings and a second cluster of embeddings 424, although there may be additional cluster(s) determined by the clustering algorithm. Once the ML model is trained, the ML model may project an epigenetic profile to a location in embedding space that is associated with a particular cluster, according to techniques discussed herein. For example, as discussed herein, an indication may be received identifying a cluster as being associated with a valid epigenetic profile, invalid epigenetic profile, invalid epigenetic profile that may be recovered using a pre and/or post-processing operation, invalid epigenetic profile that contains some valid epigenetic data, etc.

After training is finished or sufficiently progressed a cluster may be associated with an indication that the epigenetic profiles associated with the embeddings in the cluster are valid epigenetic profiles, invalid epigenetic profiles, invalid epigenetic profiles that may be recovered using one or more particular pre or post-processing operations, invalid epigenetic profiles that contain some valid epigenetic data, or the like. During training, the status of the epigenetic profiles associated with a cluster may be unknown without additional operations discussed herein.

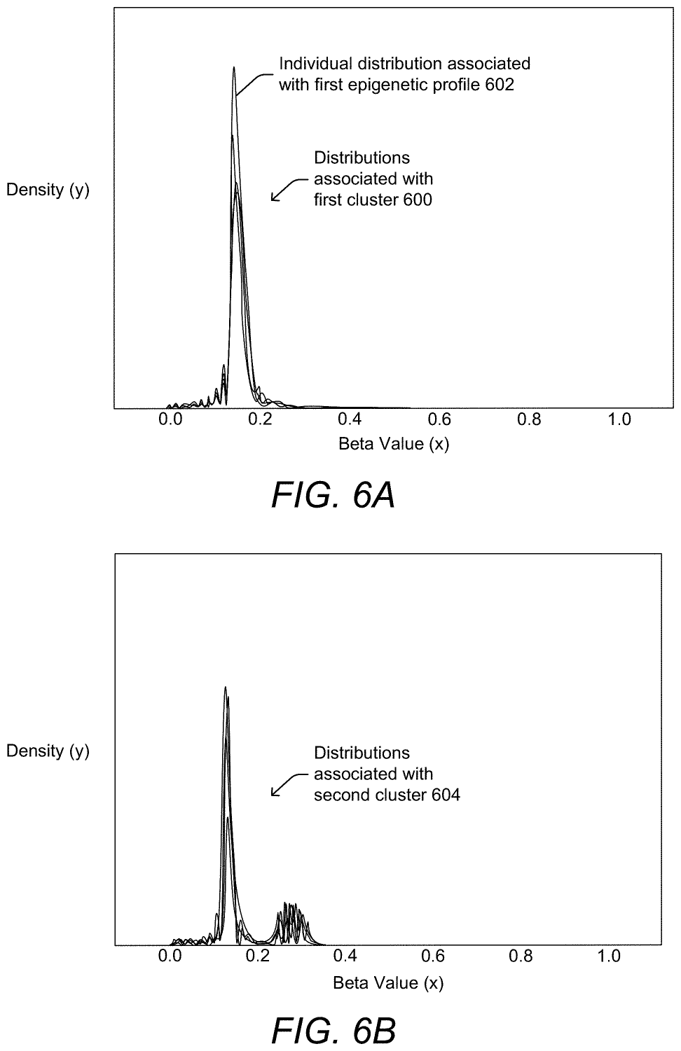

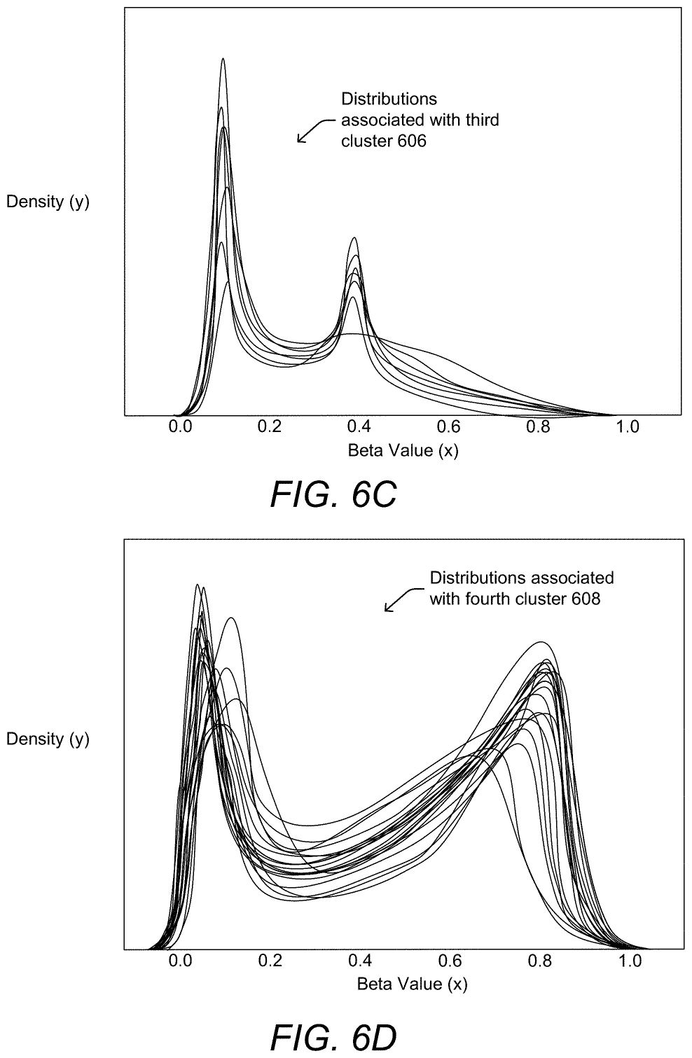

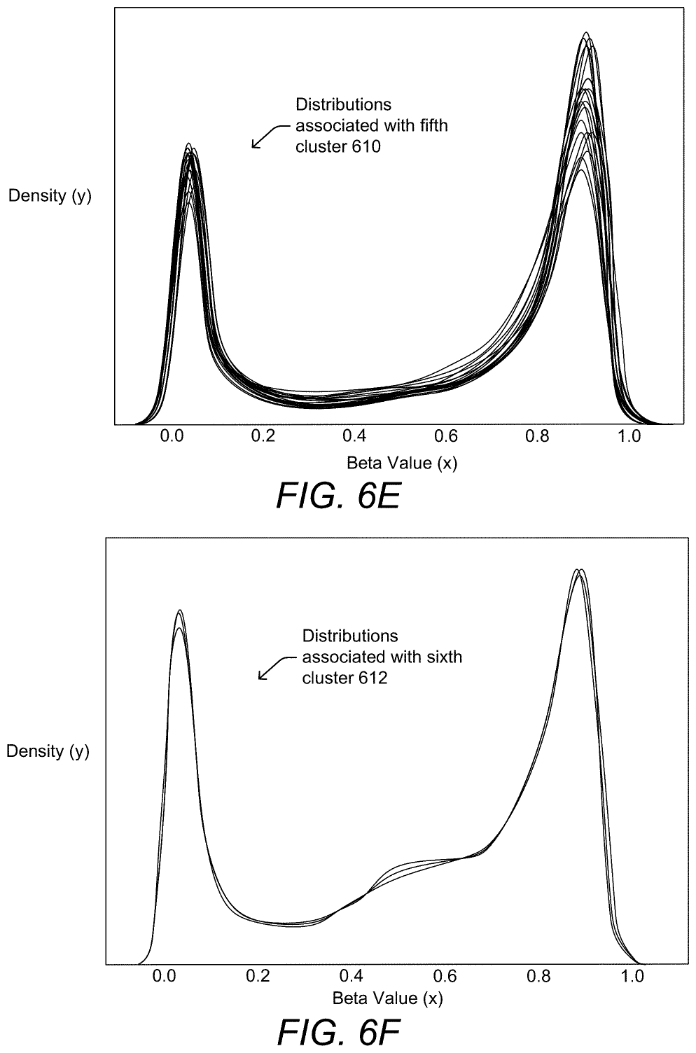

At operation 426, example process 400 may comprise causing display of distributions associated with embeddings of a first cluster, according to any of the techniques discussed herein. FIGS. 6A-6F and FIG. 7 depict examples of such displays, as discussed in more detail below. In some examples, causing the display may comprise transmitting instructions to a computing device, the instructions causing the computing device to render a graphical user interface (GUI) including a depiction of epigenetic profiles associated with at least one of the clusters. In some examples, the depiction of the epigenetic profiles may overlay the epigenetic profiles associated with a same cluster. In some examples, once the ML model is trained, operation 426 may be skipped although, in some examples, operation 426 may be retained so that the accuracy of the ML model's output may be verified. Operation 426 may additionally or alternatively comprise causing display of up to all of the clusters that were determined at operation 420. In some examples, operation 426 may be caused via communication via an API. For example, the instructions for causing the display may be transmitted via the API in response to a prior call to the API that may have provided the epigenetic profiles or identified the epigenetic profiles (e.g., where the epigenetic profiles are stored in a memory accessible to computing device(s) that are executing example process 400).

At operation 428, example process 400 may comprise receiving an indication that the epigenetic profiles associated with the first cluster are valid or invalid, according to any of the techniques discussed herein. For example, the indication may be received based at least in part on causing the display at operation 426. In some examples, the indication may be received responsive to a user selection at a GUI presented by a computing device. In an example where the ML model has already been trained, the first cluster may already be associated with such an indication. In addition to indicating that an epigenetic profile is valid or invalid, the indication may indicate a subset of DNA loci that are valid or invalid, a pre or post-processing technique for recovering invalid epigenetic data associated with one or more DNA loci, and/or a fault or condition associated with the collection, storage, and/or processing of a biological sample to determine the epigenetic profile. If the indication indicates that the first cluster is valid, example process 400 may transition to operation 430. Otherwise, if the indication indicates that the first cluster is invalid, example process 400 may transition to operation 432.

At operation 430, example process 400 may comprise using and/or passing the epigenetic profile(s) associated with the first cluster, according to any of the techniques discussed herein. Operation 430 may comprise using the epigenetic profile(s) associated with the first cluster based at least in part on passing the epigenetic profile(s) associated with the first cluster. Passing the epigenetic profile(s) may comprise transmitting the epigenetic profile(s) to a component downstream from the QC pipeline, transmitting an indication to preserve or use the epigenetic profile(s), indicating that the epigenetic profile(s) have passed the quality control process, and/or the like. Using the epigenetic profile(s) may comprise operations 118 and/or 122 and/or using the epigenetic profile(s) as part of scientific research, a medical diagnosis, or a medical treatment plan.

At operation 432, example process 400 may comprise transmitting a notification and/or excluding epigenetic data from use, and/or re-, pre-, and/or post-processing epigenetic data associated with an invalid epigenetic profile, according to any of the techniques discussed herein. Transmitting a notification may comprise exposing the notification via an API to a computing device associated with processing biological samples, a computing device associated with a subject that provided a biological sample associated with an invalid epigenetic profile, and/or the like. The notification may comprise instructions for causing a display for a GUI indicating such information and/or instructions for causing an action to be taken regarding the epigenetic profile (e.g., excluding the epigenetic profile from use, identifying a subset of the epigenetic profile that may be used, re-processing the biological sample or the epigenetic profile using a different method or a different algorithm, pre- and/or post-processing the epigenetic profile).

In some examples, re-processing the epigenetic profile may comprise repeating at least one of the operations to prepare and/or process the biological sample or epigenetic profile for use. Re- and/or pre-processing the epigenetic profile may comprise using a different biological sample acquisition, storage, or processing method (e.g., acquiring a different tissue, using a different acquisition device or solution, using a different DNA extraction or purification method, redoing the bisulfite conversion of extracted DNA, more or less aggressive cleaning and/or using alternative algorithms or quality checks to prepare the epigenetic profile, using a different biological sample processing method such as using TAPS instead of bisulfite conversion or vice versa, determining the distributions a different way (e.g., changing the number of bins, changing the range associated with one or more bins, changing the shape representing the distribution), etc.). Operation 432 may additionally or alternatively comprise obtaining a new biological sample, transmitting a notification to advise a subject of the need for a new biological sample and/or a shipping identifier associated with a biological sample collection device that will be shipped to the subject, and/or transmitting instructions to cause shipment of a biological sample collection device (e.g., transmitting instructions for a computing device to cause a biological sample collection device to be packaged, a shipping label to be printed and/or affixed to the package, etc. and/or transmitting instructions to a shipper's computing device notifying the shipper of a new package for shipping).

Post-processing the epigenetic profile may comprise transforming the data to account for a skew (e.g., multiplying by a scalar, adding a constant), applying an ML model to the epigenetic profile to recover a portion of the epigenetic profile (e.g., the ML model may be trained to receive at least a portion of the epigenetic profile as input and to output epigenetic data associated with one or more DNA loci that were associated with invalid epigenetic data, as discussed in more detail in U.S. patent application Ser. No. 16/591,296, filed Oct. 2, 2019), determining a portion of the epigenetic profile that is valid and passing/using that portion (e.g., by determining a first subset of DNA loci associated with values in the distribution that fall at or below a lower threshold (e.g., a beta-value equal to or less than 0.1, 0.15, 0.09, or the like) and/or a second subset of DNA loci associated with values in the distribution that fall at or above an upper threshold (e.g., a beta-value equal to or great than 0.9, 0.85, 0.91, or the like), etc. In some examples, a component of the quality control pipeline may conduct the re-, pre-, and/or post-processing. The component may be hardware, software, or some combination thereof (e.g., an FPGA).

In some examples, operations 402, 406, 416, 420, 427, 428, 430, and/or 432 may be used as at least part of a training algorithm for training an ML model to receive an epigenetic profile and classify the epigenetic profile as being valid, invalid, and/or invalid and a processing operation associated therewith (e.g., a subset of the epigenetic profile that is valid, a re-, pre-, or post-processing step that may recover at least some of the invalid data). For example, operations 426 and/or 428 may be at least part of a supervision operation for training the ML model. In some examples, example process 40 may be executed by the ML model as it is trained or after it is trained. For example, the ML model may comprise one or more ML models trained and configured to execute operations 402, 406, and/or 416 and to generate an output associated with operation(s) 430 and/or 432. In at least one example, a first ML model (e.g., a manifold learning projection algorithm) may determine the embeddings at operation 416 and a second ML model may cluster the embeddings output by the first ML model. During training, in an example where the number of clusters is not set as a parameter of the second ML model (e.g., using HDBSCAN), supervision may be used to disambiguate whether a cluster is associated with valid, invalid, and/or a recoverable invalid epigenetic profile. After training is complete, the different clusters output by the ML model may be known—in other words, a first one or more clusters may be associated with valid epigenetic profiles, a second one or more clusters may be associated with invalid epigenetic profiles, and a third one or more clusters may be associated with invalid epigenetic profiles associated with a recovery technique (e.g., re-, pre-, and/or post-processing operation(s)). In some examples, the training may comprise merging clusters to reduce the number of clusters generated.

In some examples, example process 400 and/or portions thereof may be done automatically, without human input required, which may comprise actuating a machine to re-process a biological sample, notifying a shipping company of a package to be picked up, printing and/or affixing a shipping label, and/or the like. Similarly, the re-, pre-, and/or post-processing may occur automatically without human involvement.

### Example Embeddings and Clusters

FIG. 5 illustrates a depiction of the embeddings (or a projection of the embeddings) 418 determined as a part of the example process of FIG. 4 and the two example clusters previously discussed, cluster 422 and cluster 424, along with additional clusters cluster 500, cluster 502, cluster 504, cluster 506, and cluster 508. Note that, although the clusters are identified in the drawings by a circle identifying a region, additional or alternate means may be used to identify cluster membership. For example, an epigenetic profile may be associated with an identifier associated with a cluster, the epigenetic profile may be added to a data structure associated with a cluster, and/or the like. In some examples, and as discussed further regarding FIG. 7, the techniques discussed herein may comprise supervising the embedding and/or clustering techniques based at least in part on changing the cluster to which an epigenetic profile belongs. For example, this may comprise moving an embedding in the embedding space or moving an embedding projection in a projected space. In an additional or alternate example, the supervision may include changing a cluster identifier associated with an embedding (and epigenetic profile associated therewith). Regardless, any of the modifications discussed above may be used to modify the embedding and/or clustering algorithm. For example, the embedding algorithm may be modified so that the embedding algorithm would generate an embedding that is closer to the location to which an embedding was moved for an epigenetic profile that is the same or similar to the embedding that was moved. Additionally or alternatively the embedding algorithm may be modified to generate an embedding in a region of the embedding space associated with a cluster to which an embedding was moved or to which the embedding was re-assigned. In other words, a similar or same epigenetic profile would be mapped to a region of the embedding space associated with the cluster to which the similar or same epigenetic profile is mapped.

FIGS. 6A-6F illustrate different clusters of distributions of epigenetic profiles. FIGS. 6A-6F may be part of a display caused by operation 426. For example, any one or more of FIGS. 6A-6F may be displayed via a GUI, such as the example GUI depicted in FIG. 7. In some examples, displaying FIGS. 6A-6F may be part of a method for training the ML model(s) discussed herein and/or for verifying classification of epigenetic profiles as valid, invalid, or invalid and at least partially recoverable. Each of FIGS. 6A-6F depicts beta values between 0.0 and 1.0 on the x-axis and the density of probes having such a beta value on the y-axis).

FIG. 6A depicts distributions (e.g., as determined at operation 406) associated with a first cluster (e.g., as determined at operation 420) 600. FIG. 6A also indicates an individual distribution associated with a first epigenetic profile 602. The distributions of the first cluster depicted in FIG. 6A are examples of distributions associated with invalid epigenetic profiles.

FIG. 6B depicts distributions associated with a second cluster 604 of embeddings. These distributions are similarly associated with invalid epigenetic profiles.

FIG. 6C depicts distributions associated with a third cluster 606 of embeddings. These distributions are associated with invalid epigenetic profiles.

FIG. 6D depicts distributions associated with a fourth cluster 608 of embeddings. These distributions are associated with invalid epigenetic profiles. However, in some cases, FIG. 6D may include one or more distributions that may comprise valid epigenetic data and/or invalid epigenetic data that may be recovered by re-, pre-, and/or post-processing the epigenetic profile or a portion thereof.

FIG. 6E depicts distributions associated with a fifth cluster 610 of embeddings. These distributions are associated with valid epigenetic profiles.

FIG. 6F depicts distributions associated with a sixth cluster 612 of embeddings. These distributions are associated with epigenetic profiles that may be valid and/or may contain some invalid epigenetic data. The techniques discussed herein may comprise filtering, using a band filter, a distribution to identify DNA loci associated with invalid epigenetic data and/or, using a high and/or low pass filter, to identify DNA loci associated with valid epigenetic data. For example, the band filter may be designed to pass a “middle” portion of the distributions identifying DNA loci that contributed to the density above 0.15 or 0.2 and below 0.8 or 0.95, for example (although other corner values may be used). The high and/or low pass filters may identify the inverse portions of the distributions.

### Example Graphical User Interface (GUI)

FIG. 7 depicts an example graphical user interface (GUI 700) for causing operations associated with different distributions generated from epigenetic profiles. A computing device may display GUI 700 based at least in part on computer-executable instructions generated at operation 426. In some examples, GUI 700 may be used as part of a training algorithm for training the ML model discussed herein. In an additional or alternate example, the GUI 700 may be part of the quality control pipeline discussed herein.

The GUI 700 may comprise display(s) of the distributions associated with one or more clusters of embeddings, as depicted in more detail in FIGS. 6A-6F, and one or more actuatable elements. The actuatable elements may be selectable by a user to cause the computing device to take one or more actions. For example, the GUI 700 may comprise an element 702 for indicating that a cluster is associated with valid epigenetic profiles, an element 704 for indicating that a cluster is associated with invalid epigenetic profiles, and/or an element 706 for indicating a fault or condition associated with one or more epigenetic profile(s). For example, a user may indicate the depicted cluster as being valid or invalid using element 702 and/or 704 and/or may provide additional data using element 706, such as a fault associated with collection, preservation, or processing of a biological sample, if one is known. Additional or alternate methods of providing label data may be used to train the ML model to detect the fault or condition associated with an invalid epigenetic profile—for example, a subject may provide information via a computing device associated with the subject regarding a time at which the subject last ate, drank, brushed their teeth, etc. before providing a biological sample and/or a health history associated with the subject may be used as label data.

The techniques discussed herein may comprise determining an estimate of the validity or invalidity of a cluster. For example, the ML model may be trained to indicate that a cluster is associated with valid or invalid epigenetic profiles. Based at least in part on this output, the GUI 700 may be populated with an estimated selection of elements 702 and/or 704, which is indicated in FIG. 7 as a gray-filled element at 708 and 710. In other words, the techniques may comprise generating instructions for the GUI 700 based at least in part on the output of the ML model, which may comprise pre-selecting or making some form of indication via the GUI 700 of the model's estimate of whether the cluster is associated with valid or invalid epigenetic profiles. In some examples, a user may affirm this estimate by a proactive selection of the element 702 or 704 or may review the suggestions and affirm the suggestions after making any changes.

GUI 700 may additionally or alternatively include an element for joining two or more clusters into one cluster, element 712; an element for isolating one or more distribution from a cluster, element 714; and/or an element for moving a distribution from one cluster to another, element 716. For example, selection of element 712 may cause two or more clusters to be joined into a single cluster (e.g., associating the distributions of the two or more clusters with a same identifier, moving the distributions of the two or more clusters into a single data structure). This selection may be used to train the embedding algorithm to map epigenetic profiles that would have been mapped into two different regions in the embedding space instead into a single region or two regions that are in closer proximity. Additionally or alternatively, this selection may be used to train the clustering algorithm to cluster embeddings in the two (or more) different regions of the embedding space into a single cluster.

Selection of element 714 may permit the user to select one or more distributions for viewing and/or moving. For example, distribution 718 has been isolated for viewing distinctly from other distributions of the same cluster by graying out the other distributions. The distribution 718 may be moved from a current cluster to any of the other clusters (e.g., by selecting element 716 and selecting one of the other clusters). This move may be used as supervision for training the embedding algorithm and/or the clustering algorithm. In other words, moving a distribution between clusters and/or merging two or more clusters may be used to train the embedding model to generate embeddings that were formerly more spread out into a smaller region of the embedding space. Such training may comprise modifying one or more parameters of the embedding model to tighten the region of the embedding space into which similar epigenetic profiles as mapped.

### Example Processes

FIG. 8 illustrates a flow diagram of an example process 800 for detecting that an epigenetic profile is faulty, detecting a fault associated with the epigenetic profile, and/or taking action regarding the fault. In some examples, example process 400 may be accomplished, at least in part, by a first and second ML model (e.g., embedding algorithm and clustering algorithm), although a single ML model may be used, and example process 800 may be accomplished by the first ML model, the second ML model, and/or a third ML model. For example, the first and/or second ML model may determine the validity or invalidity of an epigenetic profile and the third ML model may determine a fault, condition, or recovery technique associated with an invalid epigenetic profile. In an additional or alternate example, the first, second, and third ML models may be combined as a single ML model during training. For example, the single ML model may be trained to output an indication that an epigenetic profile is valid, invalid, or invalid and may be recoverable by one or more re-, pre-, or post-processing techniques. For example, the indication of recovery techniques may identify specific recovery techniques, such as accounting for a skew, re-embedding an epigenetic profile using a different algorithm, or the like.

At operation 802, example process 800 may comprise receiving an epigenetic profile associated with a biological sample, according to any of the techniques discussed herein. For example, operation 802 may comprise operation 106 and operation 402.

At operation 804, example process 800 may comprise receiving an indication that the epigenetic profile is valid or invalid, according to any of the techniques discussed herein. Operation 804 may comprise receiving an output from the embedding algorithm and/or the clustering algorithm. For example, the embedding algorithm may receive the epigenetic profile and map it to an embedding and the embedding may be classified into a cluster by the clustering algorithm. The cluster may be associated (e.g., by the training method described in FIG. 4) with an indication that the epigenetic profiles associated with that cluster are valid, invalid, or invalid and recoverable.

At operation 806, example process 800 may comprise including and/or using the epigenetic profile if the epigenetic profile is identified as being valid, according to any of the techniques discussed herein, such as operation(s) 118 and/or 122 and/or operation 430.

At operation 808, example process 800 may comprise providing as input an epigenetic profile and/or the distribution associated therewith to an ML model, according to any of the techniques discussed herein. As discussed herein, the ML model may be trained to receive the epigenetic profile and/or the distribution and map the epigenetic profile and/or the distribution to a fault associated with the collection, storage, and/or processing of a biological sample, a condition associated with the subject, and/or a successful recovery method. The ML model may be trained to detect the fault based at least in part on training data collected in association with the collection, storage, and/or processing of the biological sample. For example, the subject may provide information regarding provision of the biological sample what the subject last put in the subject's mouth and how long the subject waited before providing the biological sample, whether the subject waited to transmit the sample, whether the sample remained in a sample preservation container (e.g., to keep the sample from ultraviolet (UV) or other light exposure, heat exposure, or contamination), etc. One or more sensors, such as pressure sensor(s), thermometer(s), infrared device(s), radiation monitor(s), camera(s), etc. may provide data indicating a presence or absence of a biological sample in a collection device; a temperature of the biological sample, collection device, preservation container, or transportation unit for the preservation container; an amount of light to which the biological sample was exposed; an amount of radiation to which the biological sample was exposed at any time during collection, preservation, or processing; and/or an amount of time elapsed since the biological sample was collected or between collection of the biological sample and preservation of the biological sample (e.g., time between collection of the biological sample and placement of the biological sample in a temperature, light, and/or microbially controlled environment).

The ground truth data regarding a condition of the subject may include a health history of the subject, a smoking status associated with the subject, environmental factors associated with the subject (e.g., an air quality index, radiological value, or known environment hazard associated with a location detected by a global positioning sensor of a device of the subject at or near the time of biological sample collection), etc. The ground truth data may additionally or alternatively comprise an indication of a re-, pre-, or post-processing technique that, upon re-classification of an epigenetic profile, successfully caused the epigenetic profile to be re-classified as valid instead of invalid. For example, operation 432 may comprise re-, pre-, or post-processing an invalid epigenetic profile automatically based at least in part on the indication that the epigenetic profile is invalid and re-classifying the epigenetic profile after re-, pre-, or post-processing the epigenetic profile. If the status changes (e.g., invalid to valid) an indication may be made that the formerly invalid epigenetic profile may be associated with an indication of the recovery technique.

At operation 810, example process 800 may comprise receiving, from the ML model, an indication of a condition associated with the subject; a fault associated with the collection, preservation, or processing of the biological sample associated with the epigenetic profile; and/or a recovery technique for restoring the epigenetic data, according to any of the techniques discussed herein. In an additional or alternate example, the ML model may identify a subset of an epigenetic profile that has been identified as being invalid as containing valid epigenetic data. In some examples, U.S. patent application Ser. No. 16/591,296, filed Oct. 2, 2019, may use the subset to infer epigenetic data associated with one or more DNA loci that were indicated as being invalid.

At operation 812, example process 800 may comprise determining, based at least in part on the condition, fault, or recovery technique, instructions associated with collection, preservation, and/or processing of the biological sample, according to any of the techniques discussed herein. For example, if the there is a collection fault, the instructions may comprise generating an instruction to create a shipping label for a new collection device to be sent to the subject, transmitting a notification to a computing device associated with the subject (e.g., the notification may comprise a tracking number associated with the collection device, a notification of the faulty epigenetic data and/or fault type, directions for providing a new sample that may be specific to the fault—e.g., “Please do not use mouthwash at least two hours before providing the sample,” “Please wait to provide the sample until an hour or more after eating.”), or the like. Generating instructions for a preservation or processing fault may comprise transmitting a notification to a computing device associated with a preservation or transportation service of a preservation condition that led to the epigenetic profile being contaminated, spoiling, or the like. The instructions may additionally or alternatively cause a computing device to re-process the biological sample using an additional or alternate processing technique. In some examples, the faulty epigenetic profile may be used to detect a condition associated with the subject (e.g., previously undetected cancer may appear as invalid epigenetic profile).

At operation 814, example process 800 may comprise transmitting the notification of the condition, fault, and/or recovery technique and/or instructions to remedy the condition and/or fault to a computing device associated with the subject, a medical professional associated with the subject, a technician associated with the collection, preservation, or processing of the biological sample, or the like, according to any of the techniques discussed herein. In some examples, operation 814 may comprise causing the biological sample or epigenetic profile to be re-, pre-, or post-processed.

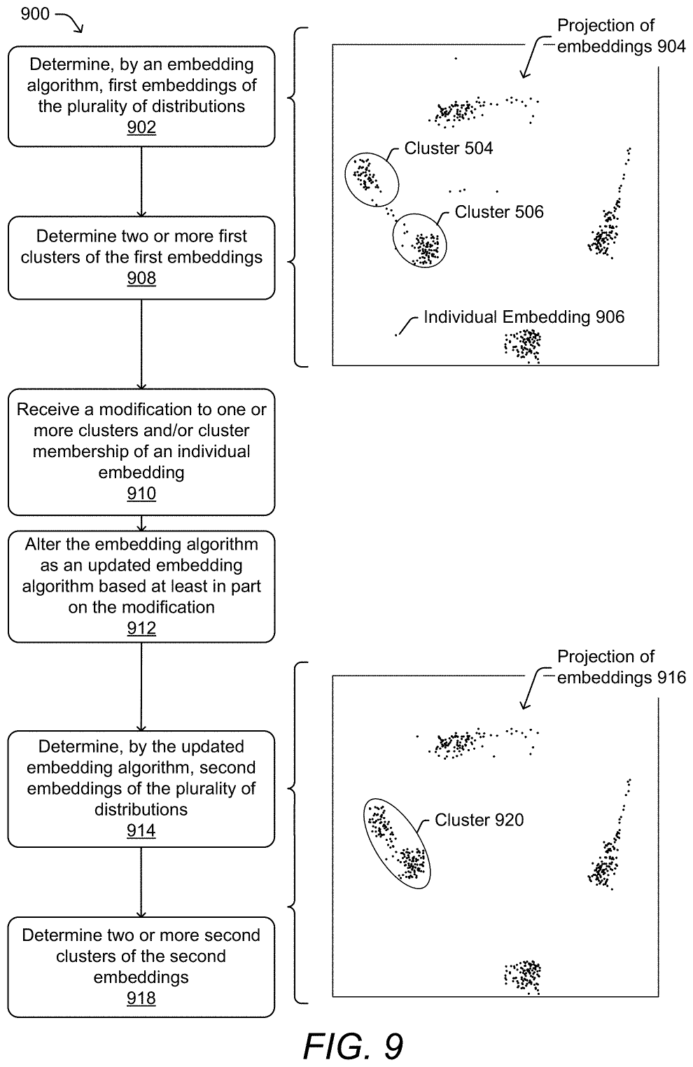

FIG. 9 depicts a pictorial flow diagram of an example process 900 for training the embedding algorithm and/or clustering algorithm.

At operation 902, example process 900 may comprise determining, by an embedding algorithm first embeddings of the plurality of distributions, according to any of the techniques discussed herein. FIG. 9 depicts projections of the first embeddings at 904. The depiction of the projections of the first embeddings includes a depiction an individual embedding 906. For the sake of example, the depicted projection of the first embeddings 904 may be the same/similar to the projection of embeddings 418.

At operation 908, example process 900 may comprise determining two or more first clusters of the first embeddings, according to any of the techniques discussed herein. For example, determining the two or more first clusters may comprise determining cluster 504 and/or cluster 506 as discussed above.

At operation 910, example process 900 may comprise receiving a modification to one or more clusters and/or a modification to the cluster membership of one or more embeddings, according to any of the techniques discussed herein. For example, a modification to one or more clusters may comprise an indication to join at least two clusters into a single cluster or dividing a single cluster into two or more clusters. In some examples, the modification may be based at least in part on instructions generated by or responsive to a selection at GUI 700. In the depicted example, a selection at GUI 700 of element 712 may indicate a computer-executable instruction to join cluster 504 and cluster 506.

In some instances, a modification to the cluster membership of an embedding may comprise indicating a cluster to associate with the embedding, which may comprise removing the embedding from a cluster, adding the embedding to a cluster, changing the cluster with which an embedding is associated to a different cluster, etc. For example, in the illustrated example, an instruction may be received (e.g., such as by selecting element 716 while isolating a particular distribution and/or by selection of the individual embedding 906 in a projection view of the embeddings, such as depicted at 904) to add individual embedding 906 to cluster 506.

In some examples, the modification may be received based at least in part on running a model to detect embeddings and/or distributions that violate a set of heuristics.

At operation 912, example process 900 may comprise altering the embedding algorithm as an updated embedding algorithm based at least in part on the modification, according to any of the techniques discussed herein. The alteration to the embedding algorithm may force or push embeddings apart, together, or towards a different cluster in the embedding space. For example, for a cluster merge modification to cluster 504 and cluster 506, the alteration may comprise modifying one or more parameters of the embedding algorithm to generate embeddings for distributions in clusters 504 and clusters 506 to be closer together in the embedding space. Contrarily, for a cluster division instruction may alter one or more parameters of the embedding algorithm to generate embeddings for distributions of a particular cluster towards disparate regions in the embeddings space. To move an individual embedding toward a first cluster, the embedding algorithm may be modified to generate an embedding for the distribution associated with the individual embedding closer to a region in the embedding space associated with the first cluster.

In an additional or alternate example, operation 912 may comprise modifying the inputs to the embedding algorithm in addition to or instead of modifying the embedding algorithm. For example, one or more distributions may be modified by appending a cluster identifier (e.g., cluster label) to the distribution. For joining two or more clusters into a single cluster a same cluster identifier may be associated with the distributions of the two or more clusters to be joined. For diving a cluster into two or more clusters, the distributions associated with the cluster may be associated with as many different cluster identifiers as there are clusters to be formed. In some examples, example process 900 may additionally or alternatively be used as a refinement block in a quality control process between operations 420 and operation(s) 430 and/or 432.

At operation 914, example process 900 may comprise determining, by the updated embedding algorithm, second embeddings of the plurality of distributions, according to any of the techniques discussed herein. Operation 914 may result in a projection of the second embeddings 916 as depicted in FIG. 9. Note that the embeddings associated with cluster 504 and cluster 506 are closer and individual embedding 906 is no longer isolated outside a cluster or being clustered by itself/with few other embeddings.

At operation 918, example process 900 may comprise determining two or more second clusters of the second embeddings, according to any of the techniques discussed herein. For example, clustering the second embeddings may result in cluster 920 (among other potential clusters). Cluster 920 may comprise distributions that were associated with cluster 504 and/or cluster 506.

In some examples, iteratively applying example process 900 may be used to refine the embedding algorithm and/or clustering algorithm such that, ultimately, the embedding algorithm outputs embeddings that fall into a region in the embedding space associated with valid samples, faulty samples, or faulty samples and a known fault associated with the faulty samples.

### CONCLUSION

Although the subject matter has been described in language specific to structural features and/or methodological acts, it is to be understood that the subject matter defined in the appended claims is not necessarily limited to the specific features or acts described. Rather, the specific features and acts are disclosed as example forms of implementing the claims.

The components described herein represent instructions that may be stored in any type of computer-readable medium and may be implemented in software and/or hardware. All of the methods and processes described above may be embodied in, and fully automated via, software code components and/or computer-executable instructions executed by one or more computers or processors, hardware, or some combination thereof. Some or all of the methods may alternatively be embodied in specialized computer hardware.

Conditional language such as, among others, “may,” “could,” “may” or “might,” unless specifically stated otherwise, are understood within the context to present that certain examples include, while other examples do not include, certain features, elements and/or steps. Thus, such conditional language is not generally intended to imply that certain features, elements and/or steps are in any way required for one or more examples or that one or more examples necessarily include logic for deciding, with or without user input or prompting, whether certain features, elements and/or steps are included or are to be performed in any particular example.

Conjunctive language such as the phrase “at least one of X, Y or Z,” unless specifically stated otherwise, is to be understood to present that an item, term, etc. may be either X, Y, or Z, or any combination thereof, including multiples of each element. Unless explicitly described as singular, “a” means singular and plural.

Any routine descriptions, elements or blocks in the flow diagrams described herein and/or depicted in the attached figures should be understood as potentially representing modules, segments, or portions of code that include one or more computer-executable instructions for implementing specific logical functions or elements in the routine. Alternate implementations are included within the scope of the examples described herein in which elements or functions may be deleted, or executed out of order from that shown or discussed, including substantially synchronously, in reverse order, with additional operations, or omitting operations, depending on the functionality involved as would be understood by those skilled in the art.

In the description of examples, reference is made to the accompanying drawings that form a part hereof, which show by way of illustration specific examples of the claimed subject matter. It is to be understood that other examples can be used and that changes or alterations, such as structural changes, can be made. Such examples, changes or alterations are not necessarily departures from the scope with respect to the intended claimed subject matter. While the steps herein can be presented in a certain order, in some cases the ordering can be changed so that certain inputs are provided at different times or in a different order without changing the function of the systems and methods described. The disclosed procedures could also be executed in different orders. Additionally, various computations that are herein need not be performed in the order disclosed, and other examples using alternative orderings of the computations could be readily implemented. In addition to being reordered, the computations could also be decomposed into sub-computations with the same results.

What is claimed is:

1. A method comprising:
receiving a set of epigenetic profiles, wherein a first epigenetic profile of the set comprises a plurality of epigenetic values associated with different DNA loci;
determining a plurality of representations associated with the set of epigenetic profiles, wherein determining the plurality of representations comprises determining a first representation associated with the first epigenetic profile;
determining, by an embedding algorithm, embeddings of the plurality of representations, wherein determining the embeddings comprises determining a first embedding associated with the first representation; and
determining, by an ML model and based at least in part on the embeddings, two or more clusters of embeddings, wherein:
a first cluster of the two or more clusters is associated with a first indication that epigenetic profiles associated with the first cluster fails a portion of a quality control pipeline, the first cluster including the first representation and one or more other representations associated with other epigenetic profiles different than the first epigenetic profile; and
a second cluster of the two or more clusters is associated with a second indication that epigenetic profiles associated with the second cluster are valid.

2. The method of claim 1, wherein at least one of the first indication or the second indication is further associated with at least one of a pre-processing operation, post-processing operation, or an instruction associated with collection, preservation, or processing of a biological sample.

3. The method of claim 1, further comprising:
providing, as input to a second ML model, at least one of the first embedding or the first representation associated with the first epigenetic profile; and
receiving, from the second ML model, a fault associated with collection, preservation, or processing of a first biological sample associated with the first epigenetic sample.

4. The method of claim 3, further comprising:
providing the first epigenetic profile as input to the ML model; and
wherein the fault is based at least in part on the epigenetic profile.

5. The method of claim 3, further comprising transmitting a notification to at least one of a first computing device associated with a subject from which a biological sample was obtained or a second computing device associated with processing the first epigenetic profile,
wherein the notification comprises instructions associated with collecting, preserving, or processing a new biological sample, the instructions being based at least in part on the fault.

6. The method of claim 3, further comprising:
receiving metadata associated with the first epigenetic sample;
receiving a second indication that a second subset of epigenetic profiles are associated with valid samples; and
training the second ML model based at least in part on the metadata and representations associated with the first epigenetic sample and the second subset of epigenetic profiles, to indicate a fault associated with an invalid sample.

7. The method of claim 5, wherein the metadata comprises at least one of:
a condition associated with an individual that provided the first epigenetic profile,
a sample collection fault,
a sample preservation fault,
a sample processing fault,
a data processing fault, or
an indication that a biological sample is acceptable.

8. The method of claim 1, further comprising:
receiving a modification to the first cluster or the second cluster, the modification including:
moving an epigenetic profile from one of the first cluster or the second cluster to the other of the first cluster or the second cluster,
joining the first cluster and the second cluster,
joining the first cluster or the second cluster to a third cluster, or
dividing the first cluster or the second cluster into two or more additional clusters;
appending a cluster label to the first representation based at least in part on the modification;
re-determining the embeddings as updated embedding based at least in part the plurality of representations and cluster labels associated with the plurality of representations; and
re-determining the two or more clusters based at least in part on the updated embeddings.

9. A system comprising:
one or more processors; and
a memory storing processor-executable instructions that, when executed by one or more processors, cause the system to perform operations comprising:
receiving a set of epigenetic profiles, wherein a first epigenetic profile of the set comprises a plurality of epigenetic values associated with different DNA loci;
determining a plurality of distributions associated with the set of epigenetic profiles, wherein determining the plurality of distributions comprises determining a first distribution associated with the first epigenetic profile;
determining, by an embedding algorithm, embeddings of the plurality of distributions, wherein determining the embeddings comprises determining a first embedding associated with the first distribution; and
determining, by an ML model and based at least in part on the embeddings, two or more clusters of embeddings, wherein:
a first cluster of the two or more clusters is associated with a first indication that epigenetic profiles associated with the first cluster fails a portion of a quality control pipeline, the first cluster including the first distribution and one or more other distributions associated with other epigenetic profiles different than the first epigenetic profile; and
a second cluster of the two or more clusters is associated with a second indication that epigenetic profiles associated with the second cluster are valid.

10. The system of claim 9, wherein the operations further comprise causing the at least one of epigenetic profiles or samples associated with the first cluster to be excluded.

11. The system of claim 9, wherein at least one of the first indication or the second indication is further associated with at least one of a pre-processing operation, post-processing operation, or an instruction associated with collection, preservation, or processing of a biological sample.

12. The system of claim 9, wherein the operations further comprise:
providing, as input to a second ML model, at least one of the first embedding or the first distribution associated with the first epigenetic profile; and
receiving, from the second ML model, a fault associated with collection, preservation, or processing of a first biological sample associated with the first epigenetic sample.

13. The system of claim 12, wherein the operations further comprise providing the first epigenetic profile as input to the ML model; and wherein the fault is based at least in part on the epigenetic profile.

14. The system of claim 12, wherein the operations further comprise transmitting a notification to at least one of a first computing device associated with a subject from which a biological sample was obtained or a second computing device associated with processing the first epigenetic profile,
wherein the notification comprises instructions associated with collecting, preserving, or processing a new biological sample, the instructions being based at least in part on the fault.

15. The system of claim 12, wherein the operations further comprise:
receiving metadata associated with the first epigenetic sample;
receiving a second indication that a second subset of epigenetic profiles are associated with valid samples; and
training the second ML model based at least in part on the metadata and distributions associated with the first epigenetic sample and the second subset of epigenetic profiles, to indicate a fault associated with an invalid sample.

16. The system of claim 9, wherein the operations further comprise:
receiving a modification to the first cluster or the second cluster, the modification including:
moving an epigenetic profile from one of the first cluster or the second cluster to the other of the first cluster or the second cluster,
joining the first cluster and the second cluster,
joining the first cluster or the second cluster to a third cluster, or
dividing the first cluster or the second cluster into two or more additional clusters;
appending a cluster label to the first distribution based at least in part on the modification;
re-determining the embeddings as updated embedding based at least in part the plurality of distributions and cluster labels associated with the plurality of distributions; and
re-determining the two or more clusters based at least in part on the updated embeddings.

17. A non-transitory computer-readable medium storing computer-executable instructions that, when executed by one or more processors, cause the one or more processors to perform operations comprising:
receiving a set of epigenetic profiles, wherein a first epigenetic profile of the set comprises a plurality of epigenetic values associated with different DNA loci;
determining a plurality of distributions associated with the set of epigenetic profiles, wherein determining the plurality of distributions comprises determining a first distribution associated with the first epigenetic profile;
determining, by an embedding algorithm, embeddings of the plurality of distributions, wherein determining the embeddings comprises determining a first embedding associated with the first distribution; and
determining, by an ML model and based at least in part on the embeddings, two or more clusters of embeddings, wherein:
a first cluster of the two or more clusters is associated with a first indication that epigenetic profiles associated with the first cluster fails a portion of a quality control pipeline, the first cluster including the first distribution and one or more other distributions associated with other epigenetic profiles different than the first epigenetic profile; and
a second cluster of the two or more clusters is associated with a second indication that epigenetic profiles associated with the second cluster are valid.

18. The non-transitory computer-readable medium of claim 17, wherein at least one of the first indication or the second indication is further associated with at least one of a pre-processing operation, post-processing operation, or an instruction associated with collection, preservation, or processing of a biological sample.

19. The non-transitory computer-readable medium of claim 17, wherein the operations further comprise:
providing, as input to a second ML model, at least one of the first embedding or the first distribution associated with the first epigenetic profile; and
receiving, from the second ML model, a fault associated with collection, preservation, or processing of a first biological sample associated with the first epigenetic sample.

20. The non-transitory computer-readable medium of claim 17, wherein the operations further comprise:
receiving a modification to the first cluster or the second cluster, the modification including:
moving an epigenetic profile from one of the first cluster or the second cluster to the other of the first cluster or the second cluster,
joining the first cluster and the second cluster,
joining the first cluster or the second cluster to a third cluster, or
dividing the first cluster or the second cluster into two or more additional clusters; and
appending a cluster label to the first distribution based at least in part on the modification;
re-determining the embeddings as updated embedding based at least in part the plurality of distributions and cluster labels associated with the plurality of distributions; and
re-determining the two or more clusters based at least in part on the updated embeddings.
